(19) United States
(12) Patent Application Publication
(10) Pub. No.: US 2007/0265495 A1
(43) Pub. Date: Nov. 15, 2007

(54) Method and apparatus for field of view tracking

(75) Inventor: Alex Vayser, Mission Viejo, CA (US)

Correspondence Address: CROCKETT & CROCKETT, 24012 CALLE DE LA PLATA, SUITE 400, LAGUNA HILLS, CA, 92653, US

(73) Assignee: Medivision, Inc.

(21) Appl. No.: 11/639,719

(22) Filed: Dec. 15, 2006

Related U.S. Application Data
(60) Provisional application No. 60/751,075, filed on Dec. 15, 2005.

(51) Int. Cl. A61B 1/045

(52) U.S. Cl. 600/109; 600/126

(57) ABSTRACT

In applications requiring remote manipulation of tools with video feedback such as surgical or dental applications, it is important to keep the instruments in the field of view. By placing trackable identification markers on one or more instruments, a video feedback system will track that instrument and keep the marked instrument in the field of view. Thus wherever the marked instrument(s) are moved, the camera will follow, keeping the marked instrument(s) in the field of view. Since instrument tracking may not always be desired, the feedback tracking system may be manually activated by a footswitch, voice control or other suitable apparatus. Alternatively, motion sensors on a users head, or cameras tracking the users eyes may be used to sense motion of the head to control the field of view.

### FIELD OF THE INVENTIONS

The inventions described below relate the field of endoscopic or microscopic surgery and more specifically to methods and apparatus for providing visual feedback to a surgeon during endoscopic or microscopic surgery

### BACKGROUND OF THE INVENTIONS

During existing endoscopic or microscopic dental, ophthalmic or surgical applications, instruments are viewed indirectly using a video feed from an endoscope, microscope or other suitable apparatus. Many times the instruments may be used outside of the field of view of the video system. When this happens, the video feedback device must be actively repositioned.

Alternatively, during advanced endoscopic surgical procedures a robotic arm may be used to hold the endoscope. The surgeon may use voice commands to actively reposition the surgical arm that supports the endoscope. This technology is certainly easier that manual manipulation of the scope, however voice control may not always be reliable. If the surgeon changes the intonation of the voice, or for example has a cold, the voice control system may not be accurate.

What is needed are methods and apparatus for automatically moving the field of view to track remote manipulation tools providing feedback to a surgeon, dentist or other user of the remote manipulation tools.

### SUMMARY

In applications requiring remote manipulation of tools with video feedback such as surgical or dental applications, it is important to keep the instruments in the field of view. By placing trackable identification markers on one or more instruments, a video feedback system will track that instrument and keep the marked instrument in the field of view. Thus wherever the marked instrument(s) are moved, the camera will follow, keeping the marked instrument(s) in the field of view. Since instrument tracking may not always be desired, the feedback tracking system may be manually activated by a footswitch, voice control or other suitable apparatus.

Instrument tracking may be accomplished by marking individual instruments with suitable passive identifiers such as colored dots or bands, or suitable active identifiers such as IR emitters. Any suitable marker may be used. Alternatively, the tracking system may be provided with a specific shape of a desired tracking target such as a tool, or some element of the surgical site. For example, a tooth may be selected as the tracking target such that if the patient moves during the procedure, the video feedback system will keep the target tooth in a fixed position in the field of view.

A suitable target tracking system may include software that will have algorithms to identify a specific image or color to track. The software will provide appropriate signals to an XYZ motorized stage or a robotic arm to maintain the relative position of the target in the field of view. In another aspect of the present disclosure, the control system may control a video acquisition or capture device and one or more lights illuminating the field of view. Thus as the instruments move, one or more lights and the video acquisition device will track the instruments providing illumination and video feedback to the user.

In another application, such as microscopy and more specifically dental microscopy, this technology could be particularly useful since the patient is awake and many times the dentist must reposition the scope. This reposition poses a significant issue since the dentist has to put the instruments down to reposition the scope.

For another example, a tool with specific color can be identified and the system can stay tracked on it. Dental instruments such as explorers, mirrors or headpieces, all can have identifying markers for tracking purposes.

During procedures where the visualization device is attached to an XYZ stage, a robotic arm or other apparatus, another technique to control the field of view may be to track the head motion of the user. By linking the motion of the users head to the motion of the video device the field of view of the surgical site may be maintained. Any suitable sensor such as inertia sensors, position sensors, ultrasonic sensors, IR or electromagnetic sensors may provide orientation data for the users head position. Head motion tracking may activated and disabled using a foot switch, voice recognition or other suitable controller.

The sensors typically output serial data that may be used to control a positioning device such as an XYZ stage or a robotic arm. So when the user turns his head to the left, the sensors through the video control apparatus will provide the necessary command to the positioning device. It may also be possible to control not just the direction but also the speed of the positioning equipment. It is also possible to use a 6-axis sensor that may more accurately capture a users body motion and translate the user motion to the robotic arm or other suitable apparatus. Motion or a combination of motions of a user may also be translated to different commands such as Zoom and focus.

In another still further aspect of the present disclosure, focus of the video acquisition element may be maintained using any suitable technique such as auto-focus or by tracking and controlling the motion of the video acquisition element to maintain a fixed distance from the operating field.

### BRIEF DESCRIPTION OF THE DRAWINGS

FIG. 1 is a block diagram of a field of view control system according to the present disclosure.

FIG. 2 is a side view of a first surgical instrument in use with a tracking marker.

FIG. 3 is a side view of a second surgical instrument in use with a tracking marker.

FIG. 4 is a side view of a third surgical instrument in use with a tracking marker.

FIG. 5 is a perspective view of a dental site with a tracking marker.

FIG. 6 is a perspective view of a dental tool in use with a tracking marker.

FIG. 7 is a side view of a dental mirror according to the present disclosure.

FIG. 8 is a side view of an alternate instrument according to the present disclosure.

FIG. 9 is a side view of another alternate instrument according to the present disclosure.

FIG. 10 is a perspective view of a head tracking system according to the present disclosure.

### DETAILED DESCRIPTION OF THE INVENTIONS

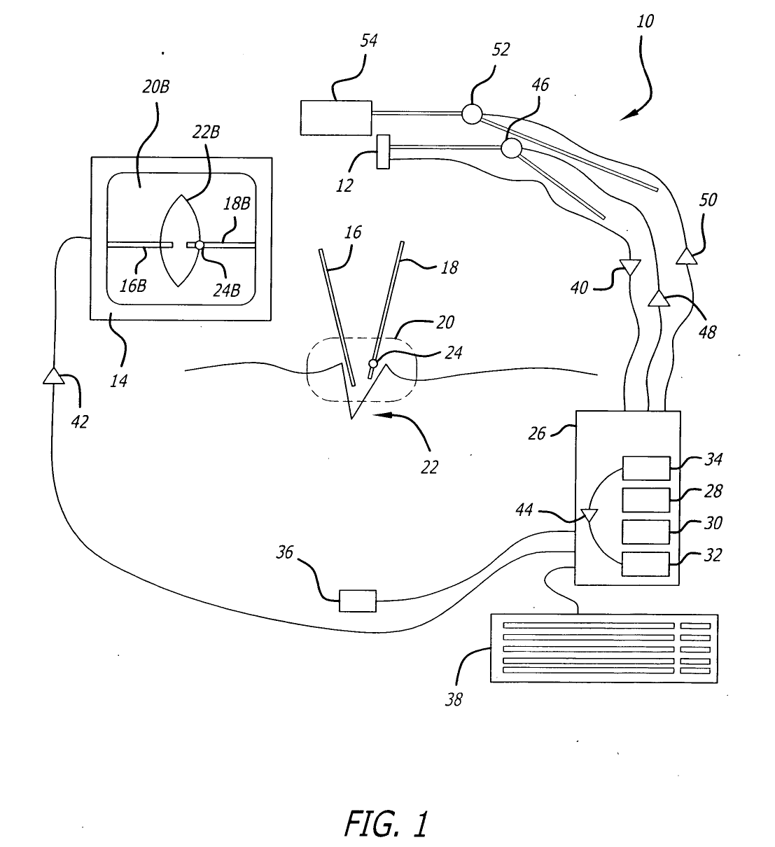

Referring to FIG. 1, field of view control system 10 includes camera 12 and display 14 for providing visual feedback to a user manipulating tools such as tool 16 and tool 18. Camera 12 has field of view 20 that may be viewed by a user as image 20B. Image 20B permits a user to view surgical site 22 and the manipulation of tools 16 and 18 within field of view 20. One or more markers such as marker 24 may be applied to a tool such as tool 18 to permit tracking controller 26 to autonomously move field of view 20 or camera 12 to keep marker 24 within field of view 20.

For the purposes of this application, field of view is the portion of a surgical site such as surgical site 22 that may be seen on a remote device such as display 14. Movement of the field of view may be accomplished by movement of one or more of the lenses or lens elements focusing light or adjacent electromagnetic radiation into the camera or its image capture element. Alternatively the image capture element may only display a portion of the image captured on a large image capture element. Thus by selecting a subset of the image capture element different areas of the surgical site.

Tracking controller 26 may be any suitable control system. Tracking controller 26 may include at least one computer processor such as computer processor 28, memory 30 and one or more user input devices such as foot switch 36 and or keyboard 38. Image data 40 from camera 12 may be processed by tracking controller 26 using software 34 to generate commands 44 necessary to move camera 12 to maintain marker 24 in field of view 20. Processed image data 40 may also result in image signal 42 that may be applied to display 14 to generate image 20B.

Commands 44 may be sent directly to a manipulation unit such as motor 46 to cause movement of camera 12. Alternatively, commands 44 may be applied to command signal generator 32 that may decode commands 44 and generate movement commands 48.

Commands 44 may also be used to generate illumination control signals 50 that may be applied to illumination motor 52 to cause movement of illumination apparatus 54.

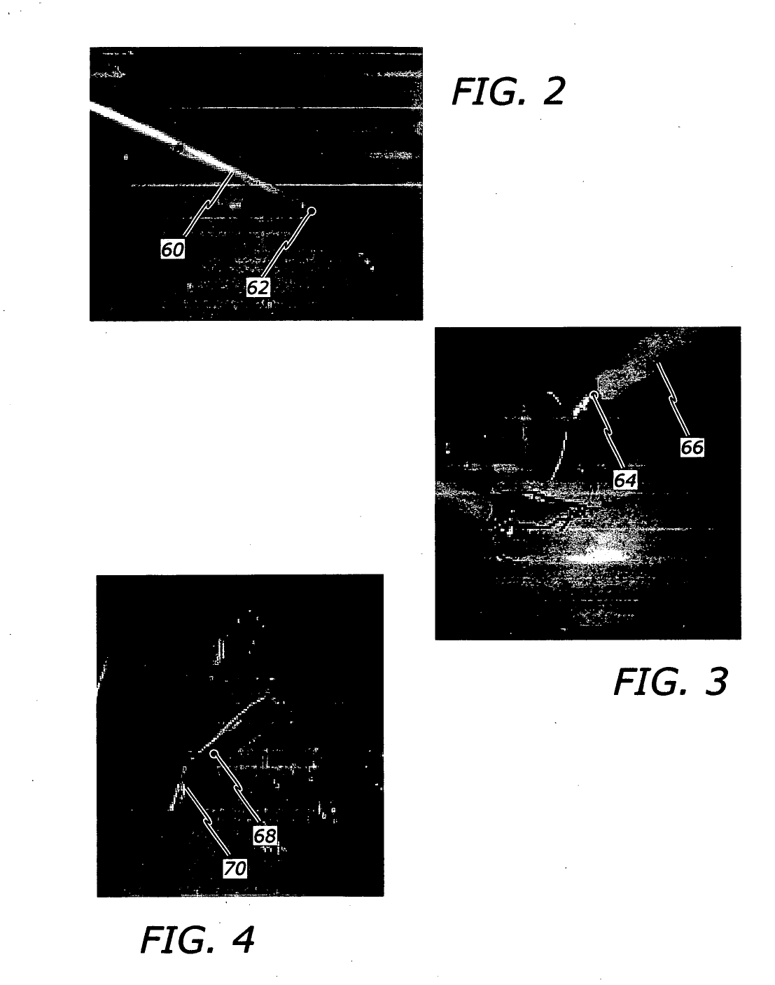

Referring now to FIGS. 2-4, various surgical sites are illustrated with tools 60, 66 and 70 carrying markers 62, 64 and 68 respectively. Any suitable size, color and shape for markers 62, 64 and 68 may be used.

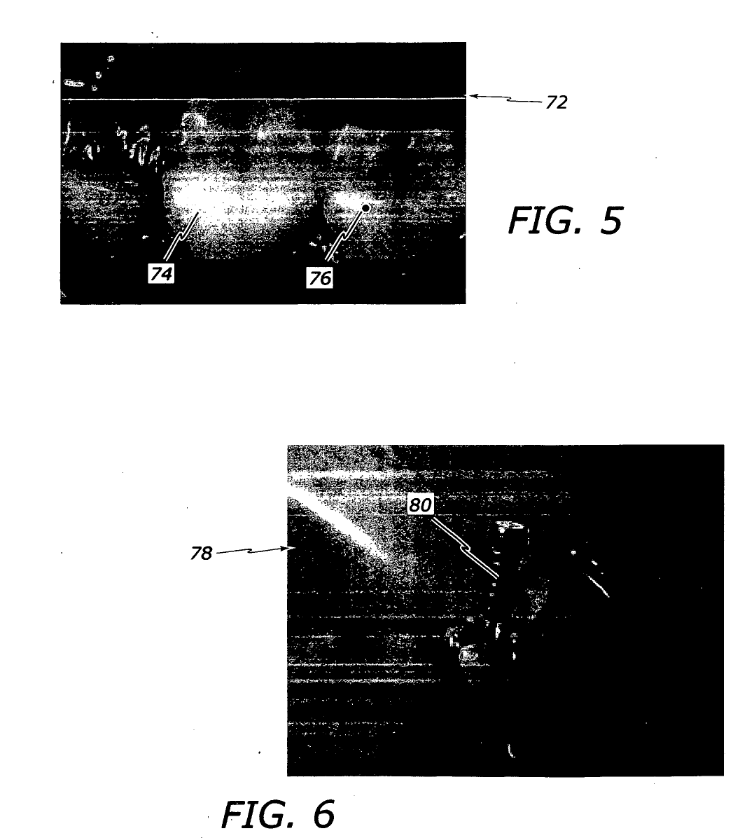

Referring now to FIG. 5, a dental site having field of view 72 may have one or more teeth such as tooth 74 in a field of view. A marker such as marker 76 may be applied to any suitable structure with as field of view such as tooth 77. Tooth 77 is adjacent to tooth of interest 74. Field of view control system may then be set to maintain marker 76 in a fixed area of the field of view such that tooth of interest 74 is maintained in the center of field of view 72.

Referring now to FIG. 6, in field of view 78 tool 80 may be identified to the control system as a field of view key or the device or mark to be maintained in the field of view. Any suitable aspect of tool 80 such as shape, color, size or other may be used to identify it as the key to the control system.

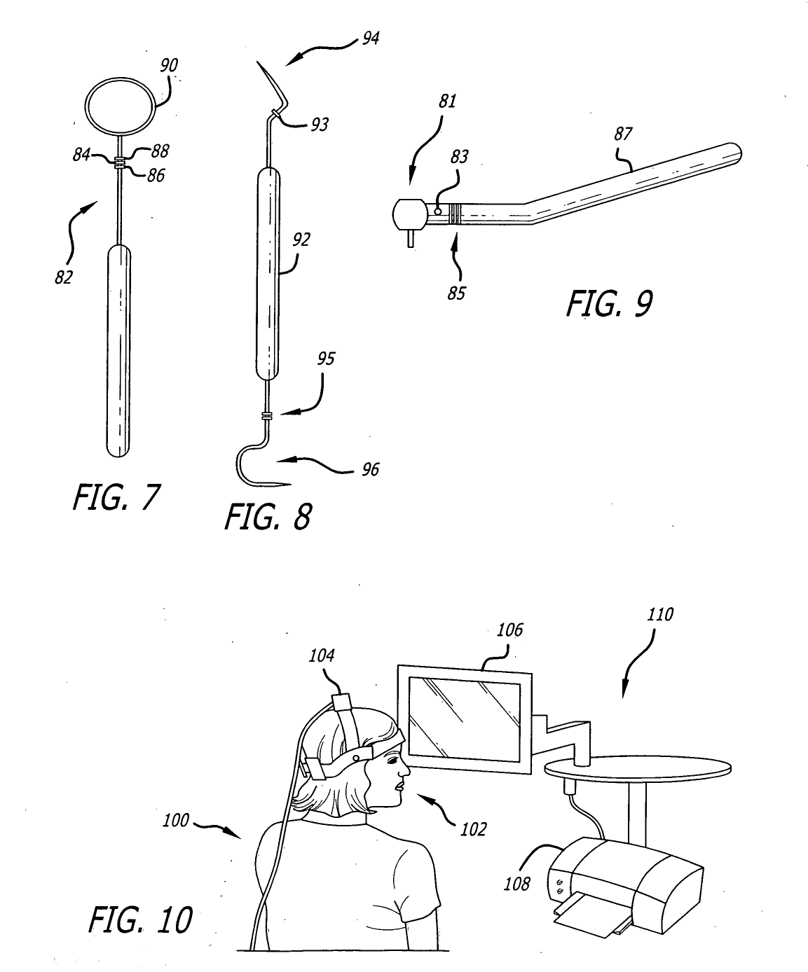

Referring now to FIG. 7, tool 82 may include one or more tracking keys such as band 84, 86 and 88. The bands may be a single color or multiple colors, or each band may have a single color and the combination of colored bands may be used as the tracking key for the tracking controller. Alternatively, the functional element of a tool may be colored or otherwise distinguishable to the tracking controller. An edge such as edge 90 of tool 82 may be colored or otherwise distinguishable.

Referring now to FIG. 8, tools such as tool 92 with two working ends such as end 94 and end 96 may also include one or more tracking keys such as key 93 and key 95. Keys 93 and 95 may distinguish between ends 94 and 96 respectively for the tracking controller.

Referring now to FIG. 9, tool 87 may include tracking key 85 or tracking key 83, or the shape of working end 81 may be used as a tracking key for the tracking controller.

Referring now to FIG. 10, field of view tracking system 110 may include tracking controller 108, view screen 106 and motion tracker 104. Motion tracker 104 may be worn on head 102 of user 100. Motion tracker 104 may detect the motion of head 102 and provide a corresponding motion of the field of view by moving a camera such as camera 12 in FIG. 1. Motion tracker 104 may be substituted by one or more cameras focused on the users eyes and translating motion of the users eyes to motion of the field of view.

Thus, while the preferred embodiments of the devices and methods have been described in reference to the environment in which they were developed, they are merely illustrative of the principles of the inventions. Other embodiments and configurations may be devised without departing from the spirit of the inventions and the scope of the appended claims.

I claim:

1. An apparatus for providing video feedback to a surgeon performing microsurgery comprising:
a camera having a field of view adapted to encompass a first portion of a surgical field;
a video monitor for viewing the field of view of the camera;
a surgical tool for use in the surgical field;
a tracking marker secured to the surgical tool;
means for moving the field of view to view another portion of the surgical field; and
means for controlling the means for moving, the controlling means autonomously keeping the tracking marker in the field of view.

2. The apparatus of claim 1 wherein the tracking marker is passive.

3. The apparatus of claim 1 wherein the tracking marker is active.

4. The apparatus of claim 1 wherein the tracking marker is the shape of the surgical tool.

5. The apparatus of claim 1 wherein the tracking marker is a badge or indicia applied to the surgical tool.

6. the apparatus of claim 1 further comprising:
one or more user input devices communicating with the controlling means.

7. The apparatus of claim 6 wherein a user input device is a foot switch for engaging or disengaging the controlling means.

8. The apparatus of claim 1 wherein the means for controlling further comprises:
a computer processor;
a memory; and
instructions for autonomously keeping the tracking marker in the field of view.

9. A method of performing microscopic surgery comprising the steps:
providing a camera having a field of view adapted to encompass a first portion of a surgical field;
providing a video monitor for viewing the field of view of the camera;
using a surgical tool in the surgical field;
providing a tracking marker secured to the surgical tool;
moving the camera autonomously to keep the tracking marker in the field of view.

10. The apparatus of claim 9 wherein the step of providing a tracking marker further comprises providing a passive tracking marker secured to the surgical tool.

11. The apparatus of claim 9 wherein the step of providing a tracking marker further comprises providing an active tracking marker secured to the surgical tool.

12. The apparatus of claim 9 further comprising the step:
providing a control means for moving the camera autonomously to keep the tracking marker in the field of view; and
wherein the tracking marker is a shape associated with the surgical tool.
